(19) United States
(12) Patent Application Publication
(10) Pub. No.: US 2002/0116056 A1
(43) Pub. Date: Aug. 22, 2002

(54) Device for controllably altering the curvature of the cornea

(76) Inventor: James F. Kirk, Gainesville, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/747,512

(22) Filed: Dec. 21, 2000

(51) Int. Cl. A61F 2/14

(52) U.S. Cl. 623/5.11; 623/906

(57) ABSTRACT

The present invention is a device that improves visual acuity by reshaping the cornea, by subjectively altering the corneal curvature in the optic zone by increasing the mechanical stiffness of the cornea outside (and/or slightly inside) the optic zone. One or more of the corneal battens are inserted into the cornea, after selectively distorting the natural corneal shape. Once inserted the stiffness of the corneal batten prevents the cornea from returning to its original curvature.

### FIELD OF THE INVENTION

The present invention relates to an apparatus and method of reshaping the cornea, and more particularly to a semi-rigid member adapted for insertion into the cornea of the eye to correct refractive errors.

### BACKGROUND OF THE INVENTION

Approximately eighty percent of the refractive power of the eye is at the cornea. As shown in FIGS. 1 and 2, the cornea is comprised of several layers. Starting on the outside (anterior) surface, there is the epithelium. This layer is ca. 6 cells thick. Beneath the epithelium is the Bowman's layer, a thin, primarily acellular collagen layer. The bulk of the mechanical strength of the cornea comes from the stroma which lies beneath the Bowman's layer. The stroma comprises the bulk of the cornea and is made of layers of oriented collagen fibers. The orientation of the fibers is very precise and the axial direction changes with each layer. On the interior (posterior) surface of the cornea are the Descemet's membrane and then the one cell thick endothelium.

The schematic eye assumes an orb of 13 mm radius on which is found a clear corneal membrane of 7.7 mm anterior radius and 6.8 mm posterior radius and a 0.55 mm center thickness (CT) in the optical zone (OZ). If the orb of the eye is truncated or elongated and/or if the radii or CT of the cornea are not of schematic dimensions, then the eye will be myopic or hyperopic. Depending on the severity of deviation from schematic dimensions, the patient will need spherical correction in the form of surgery, contact lenses, and/or spectacles to achieve maximum visual acuity.

In spherical corrections, myopic patients need to reduce the power of the cornea. This is achieved in surgery by enlarging the radius or “flattening” the curve. When treated with lenses (contact or spectacle), myopic patients have “minus” power lenses. Hyperopic patients have corneas that are too flat. Surgical treatments are aimed at decreasing the radius of curvature or “steepening” the curve. If treated with lenses, hyperopic patients wear “plus” power lenses.

Likewise, if the anterior (or posterior) radius of the corneal is not uniform as one scans around the pupillary axis (or the axis of symmetry which runs through the center of the OZ), the cornea is said to possess astigmatism. Astigmatism can also be corrected through surgery or through the use of “toric” lenses (either contact or spectacle). Regular astigmatism can be broken down into lens system in which a cylindrical lens is imposed on a spherical lens. In irregular astigmatism, the astigmatic portion of the lens system is something other than cylindrical.

A variety of means have been proposed to reshape the cornea to bring about spherical and/or stigmatic correction. These methods fall into four major classes: optical elements, stromal removal, stromal remodeling, and non-optical implants.

Optical element methods involve placing another material in the optical path, as shown in U.S. Pat. Nos.: 4,799,931; 4,851,003, 5,108,428, and 5,201,762. A class of the optical elements can be thought of as a hydrogel contact lens placed inside the cornea. In some instances, the optical element relies on a higher index of refraction for the implanted material in order to effect greater optical correction with less material. Others rely merely on the bulk of the implanted material altering the curvature sufficiently to achieve the desired correction. Either way, it is important that the material be optically transparent, capable of diffusing water, gasses, salts, and other nutrients, and non-toxic/bio-acceptable.

Stromal removal is one of the earliest methods employed to reshape the cornea. One such method involves freezing the cornea and removing a thick layer of the optic zone. This so-called “button” was placed on a lathe and stromal material was cut off using very sharp lathe tools. Other methods have proposed removing epithelial, Bowman's, and stromal tissue in situ, U.S. Pat. No. 4,834,748.

In recent years, stromal ablation has been used. Most often this is done with lasers, e.g., U.S. Pat. No. 4,988,348, but has also been proposed with radio-frequency electromagnetic radiation, e.g., U.S. Pat. No. 4,907,586. One such procedure, generally referred to as photo-refractive-keratotomy (PRK), involves the removal of the epithelial layer (with or without removing Bowman's layer) prior to ablating the stroma, as shown in U.S. Pat. Nos.: 5,269,795; 5,632,757; and 5,649,943. The epithelium takes several days to recover the entire optic zone.

Another procedure, referred to as LASIK, involves the cutting back as a thin flap the epithelial layer, Bowman's layer, and a thin layer of stroma prior to ablation of the stromal bed. The flap includes the entire optic zone. Using this flap speeds recovery since the epithelial cells do not need to regenerate. Moreover, the Bowman's layer is left largely intact.

A well-studied method of stromal remodeling is radial keratotomy (RK). In an RK procedure, 1 to 16 radial incisions (preferably fewer than 5) are made into the anterior surface of the cornea. The incisions run from the periphery (or sometimes inside) of the OZ to the limbus (the junction of the cornea with the sclera). The cuts can be as deep as 90% (or more) of the stromal thickness. The cuts weaken the cornea and permit it to sag (take on a smaller radius of curvature) outside of the OZ. This causes flattening inside the OZ. As the stromal and epithelial cells remodel the wounded area (normal wound healing), the cornea continues to change shape. This ongoing remodeling as well as the lack of a precise correlation between cut geometry/placement and final corneal shape are two major drawbacks of the RK procedure itself. Other drawbacks include corneal perforation and night (glare) blindness due to light scattering off of that portion of the incision that is inside the OZ.

Another method of stromal remodeling involves thermal denaturation of the proteins in the stroma. Collagen is the primary form of protein in the stroma and heating can change its morphology. A variety of methods have been proposed to effect denaturation of the stromal collagen, including, ultrasonic energy, U.S. Pat. No. 3,776,230, radio-frequency electromagnetic energy, U.S. Pat. No. 4,381,007, and laser, U.S. Pat. No. 5,374,265. Stromal remodeling can be performed with or without the application of a hard contact lens or other surface to mold the cornea while the stromal cells remodel the damaged collagen, U.S. Pat. Nos. 3,776,230 and 3,831,604.

A third method of stromal remodeling involves using drugs, U.S. Pat. No. 3,760,807, or enzymes, U.S. Pat. No. 5,270,051, to soften the corneal material. A rigid contact lens or similar mold surface is held against the corneal until the corneal material returns to its original mechanical strength. The mold or lens is removed and the corneal retains this new shape. It has even been proposed that this remodeling can be effected simply by holding a mold of proper shape against the cornea for a time sufficient for the cornea to remodel itself in response to the forces imposed by the mold, U.S. Pat. No. 5,695,509.

Another method of reshaping the cornea is with non-optical corneal inserts. A number of intrastromal inserts have been proposed which are to be placed just outside of the optic zone, including, U.S. Pat. Nos.: 5,733,334, 5,792,161, 5,824,086, 5,843,105, 5,855,604, 5,876,439, and 5,944,752. One groups of inserts is a split ring. Once implanted, the split ring is expanded or contracted to an extent that provides the amount of reshaping that is desired in the optic zone. The ring is then fixed in that configuration.

Another group of implants is a hollow ring. The thickness of the implanted ring is adjusted by adding or removing material from the internal void in the ring. This change in thickness alters the shape of the cornea in the optic zone.

A third group of implants is comprised of two, nearly semicircular, ring segments. One segment is implanted on each side of the optic zone. The thickness of the implant determines the extent to which the curvature of the optic zone is altered.

Notwithstanding the foregoing, there remains a need for an improved method of reshaping the cornea, which can produce substantially permanent results in a short time period.

### BRIEF SUMMARY OF THE INVENTION

The present invention is in the class of processes and devices that improve visual acuity by reshaping the cornea. Whereas previous processes and devices have been disclosed which reshaped the corneal by use of optical elements, removing stromal (and other) tissue, thickening the cornea outside the optical zone, or by weakening or otherwise damaging the stromal tissue, the present invention subjectively alters the corneal curvature in the optic zone by increasing the mechanical stiffness of the cornea outside (and/or slightly inside) the optic zone.

The present invention, referred to herein as a corneal batten, comprises minute fibers made from materials with a Young's modulus greater than the Young's modulus of the corneal stroma. One or more of the corneal battens are inserted into the cornea, after selectively distorting the natural corneal shape. Once inserted the stiffness of the corneal batten prevents the cornea from returning to its original curvature.

The corneal batten is surgically implanted into the corneal stroma. The curvature of cornea is controllably altered by the following: selection of the position of placement of the corneal battens; varying the number of corneal battens implanted; varying the dimensions of implanted corneal battens; and by flattening or over curving the corneal during placement of the corneal battens. The inserted corneal battens will act to alter the original curvature of the cornea and bring about improved visual acuity.

The radius of curvature of the optic zone is decreased by inserting at least one corneal batten between the layers of the corneal stroma in the limbal region. In this case, the long axis of the corneal batten is oriented radially to the center of the cornea, i.e., the pupillary axis.

Alternatively, the radius of curvature of the optic zone is decreased by inserting at least one corneal batten so as to penetrate several layers of the corneal stroma in the limbal region. In this case, the long axis of the corneal batten is oriented tangentially to the center of the cornea, i.e., perpendicular to the pupillary axis.

The radius of curvature of the optic zone is increased by inserting at least one corneal batten so as to penetrate several layers of the corneal stroma in the limbal region. The long axis of the corneal batten is oriented radially to the center of the cornea, the pupillary axis.

Alternatively, the radius of curvature of the optic zone is increased by inserting at least one corneal batten between layers of the corneal stroma in the limbal region. The long axis of the corneal batten is oriented tangentially to the center of the cornea, perpendicular to the pupillary visual axis.

These and other objects, features and advantages of the present invention will be more readily understood with reference to the following detailed description, read in conjunction with the accompanying drawing figures.

All patents referred to or cited herein are incorporated by reference in their entirety to the extent they are not inconsistent with the explicit teachings of this specification, including: U.S. Pat. No. 3,760,807 (Neefe), U.S. Pat. No. 3,776,230 (Neefe), U.S. Pat. No. 3,831,604 (Neefe), U.S. Pat. No. 4,381,007 (Doss), U.S. Pat. No. 4,799,931 (Lindstrom), U.S. Pat. No. 4,834,748 (McDonald), U.S. Pat. No. 4,851,003 (Lindstrom), U.S. Pat. No. 4,907,586 (Bille, et al.), U.S. Pat. No. 4,988,348 (Bille), U.S. Pat. No. 5,002,571 (O'Donnell, Jr., et al.), U.S. Pat. No. 5,063,942 (Kilmer et al.), U.S. Pat. No. 5,108,428 (Capecchis, et al.), U.S. Pat. No. 5,201,762 (Huber), U.S. Pat. No. 5,269,795 (Arnott), U.S. Pat. No. 5,270,051 (Harris), U.S. Pat. No. 5,318,044 (Kilmer et al.), U.S. Pat. No. 5,368,604 (Kilmer et al.), U.S. Pat. No. 5,374,265 (Sand), U.S. Pat. No. 5,395,385 (Kilmer et al.), U.S. Pat. No. 5,632,757 (Arnott), U.S. Pat. No. 5,649,943 (Amoils), U.S. Pat. No. 5,695,509 (El Hage), U.S. Patent No. 5,733,334 (Lee), U.S. Pat. No. 5,766,171 (Silvestrini), U.S. Pat. No. 5,776,192 (McDonald), U.S. Pat. No. 5,779,696 (Berry et al.), U.S. Pat. No. 5,788,957 (Harris), U.S. Pat. No. 5,792,161 (de Almeida Cunha), U.S. Pat. No. 5,824,086 (Silvestrini), U.S. Pat. No. 5,843,105 (Mathis et al.), U.S. Pat. No. 5,855,604 (Lee), U.S. Pat. No. 5,876,439 (Lee), U.S. Pat. No. 5,891,131 (Rajan et al), U.S. Pat. No. 5,932,205 (Wang et al.), U.S. Pat. No. 5,934,285 (Kritzinger et al.), U.S. Pat. No. 5,944,752 (Silvestrini), and U.S. Pat. No. 6,066,170 (Lee).

### BRIEF DESCRIPTION OF THE DRAWINGS

FIG. 1 is a schematic illustration of a horizontal section of the eye.

FIG. 2 is a detailed schematic illustration of a horizontal section of the frontal portion of the eye showing the various layers of the cornea.

FIG. 3 is a perspective view of the corneal batten.

FIG. 4a is a cross sectional view of the corneal batten having a circular cross section.

FIG. 4b is a cross sectional view of the corneal batten having a hollow circular cross section.

FIG. 5a is a cross sectional view of the corneal batten having an elliptical cross section.

FIG. 5b is a cross sectional view of the corneal batten composed of seven smaller fibers twisted together and bonded with a biologically acceptable coating

FIG. 6 is a schematic illustration of a horizontal section of the eye having a corneal batten implanted radially to the center of vision.

FIG. 7 is a top view of the eye having a corneal batten implanted radially to the center of the cornea.

FIG. 8 is a schematic illustration of a horizontal section of the eye having a corneal batten implanted tangent to the optic zone.

FIG. 9 is a top view of the eye having a corneal batten implanted tangent to the optic zone.

FIG. 10 is a side view of the frontal portion of the eye having corneal batten inserted between the layers of the corneal stroma.

FIG. 11 is a side view of the frontal portion of the eye having corneal batten inserted through multiple layers of the corneal stroma.

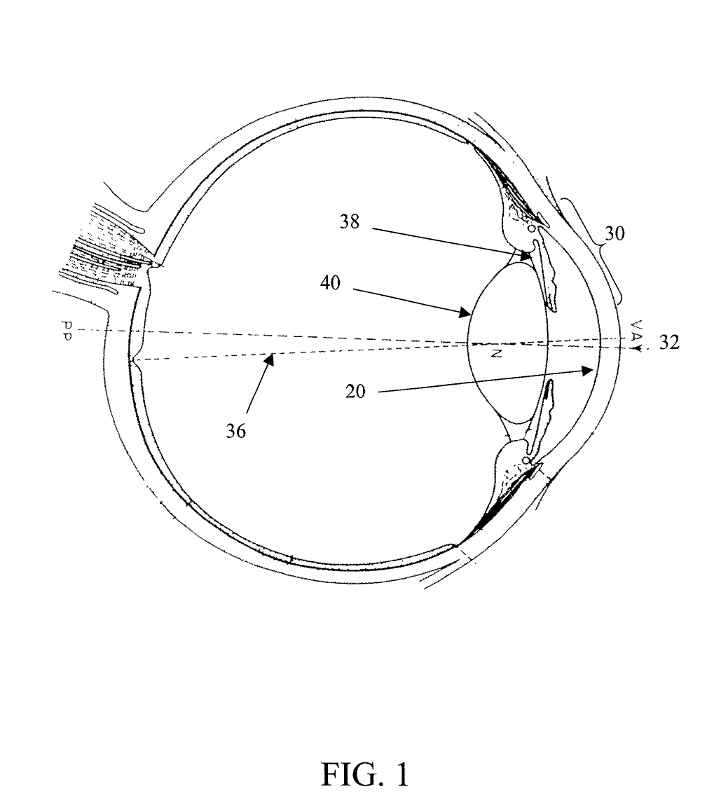

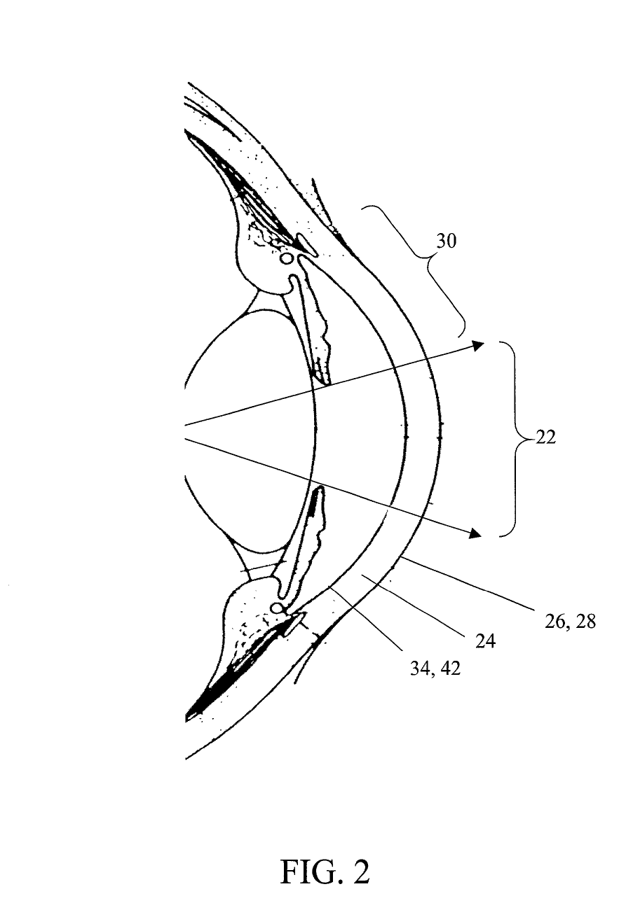

### DETAILED DISCLOSURE OF THE INVENTION

The present invention provides an apparatus and method for reshaping the cornea 20 to correct refractive error associated with near sightedness, far sightedness, and astigmatism.

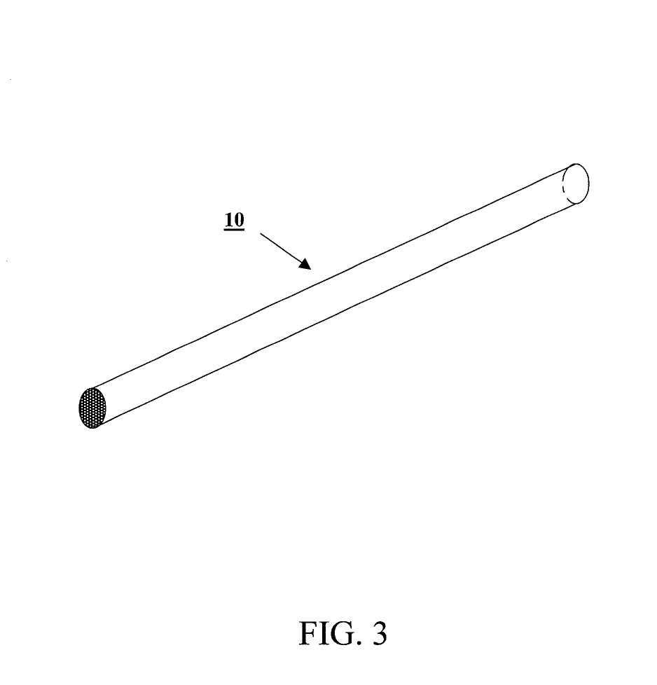

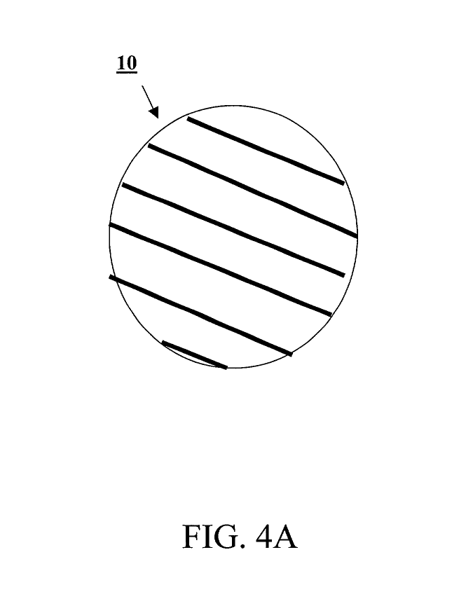

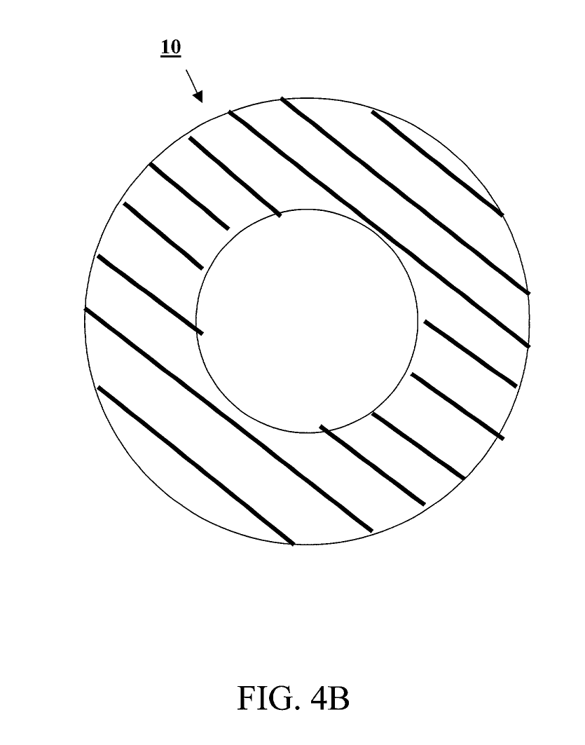

In an embodiment of the present invention, as shown in FIGS. 3 and 4a, the corneal batten 10 comprises minute fibers made from materials with a Young's modulus greater than the Young's modulus of the corneal stroma 24. One or more of the corneal battens 10 are inserted into the cornea 20, after selectively distorting the natural corneal shape. Once inserted the stiffness of the corneal batten 10 prevents the cornea 20 from returning to its original curvature.

In an embodiment, the corneal batten 10 is about 0.1 mm to 16 mm in length, preferably being about 2 mm to 6 mm in length. The cross sectional dimension of the corneal batten 10 is about 0.1 microns to 300 microns, preferably having a cross section dimension of about 1.0 microns to 25 microns. Materials having a higher Young's modulus of the material, enable the corneal batten 10 to have the smaller dimensions.

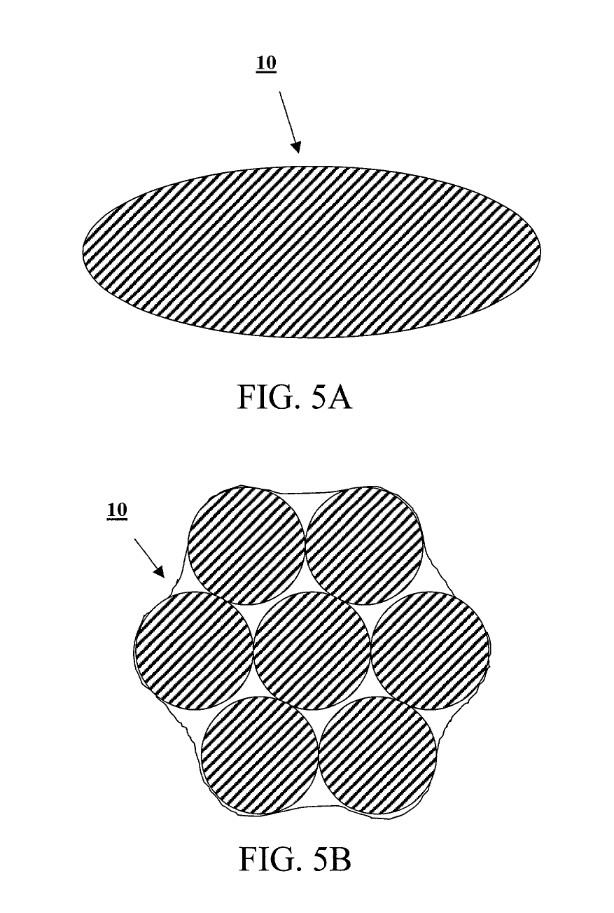

In a preferred embodiment, the corneal batten 10 has a substantially circular cross section. Alternatively, as shown in FIG. 5A, the corneal batten's 10 cross section can be elliptical, rectangular, triangular, etc. As well, the corneal batten 10 can possess a hollow cross section, as shown in FIG. 4b.

In an example, the applied load required to take a 4 mm diameter circular section of the optic zone 22 from ca. 7.7 mm radius of curvature to flat (infinite radius of curvature) equates to an applied pressure that is ca. 1.25 times the intraocular pressure (IOP). As shown in Table 1., graphite corneal battens 10 of circular cross section and of the length and diameters shown require uniformly distributed loads ranging from 0.1 to 51.6 times IOP in order to be deflected from straight to a radius of curvature of ca 7.7 mm. It is possible for relatively small implants to exert force on surrounding tissue equal to or well in excess of the predominant source of load, namely IOP. In general, the stiffness of a fiber of circular cross section varies inversely with the cube of the fiber length and proportionately with the 4^{th} power of the fiber diameter. For any given material modulus, small changes in the fiber length or fiber diameter will permit the construction of a corneal batten with the stiffness appropriate for the desired application.

TABLE 1
Multiples of the IOP (18 mm HG assumed) needed to
deflect a graphite fiber of circular cross section and of
the dimension shown from straight to ca. 7.7 mm radius of
curvature (Young's modulus of 379 × 10⁹ Pascal assumed).

| Fiber | Diameter of fiber in microns | | |
|---|---|---|---|
| Length (mm) | 7.5 | 15 | 20 |
| 2.0 | 1.0 | 16.3 | 51.6 |
| 4.0 | 0.3 | 4.0 | 12.7 |
| 6.0 | 0.1 | 1.8 | 5.7 |

To provide −3.00 diopters of power shift to the cornea would require flattening the schematic cornea from 7.70 mm radius of curvature on the anterior surface to 8.26 mm radius of curvature. The less the change in radius, the fewer corneal battens 10 will be needed to bring about the change.

In another embodiment, the corneal batten 10 is made from a bio-compatible material, acceptable to the ocular environment, having a Young's modulus greater than that of the corneal stroma 24. Such bio-compatible material can include, but is not limited to, poly-methylmethacrylate (PMMA), graphite, aluminum oxide, silicon nitride, or silicon carbide.

In an alternative embodiment, the corneal batten 10 is made from a bio-compatible composite material, acceptable to the ocular environment, having a Young's modulus greater than that of the corneal stroma 24. Such bio-compatible composite materials can include, but are not limited to, PMMA containing graphite fibers or carbon nanotubes, BIOGLASS containing aluminum oxide fibers, graphite fibers or carbon nanotubes. A schematic representation of one possible embodiment of such a composite is shown in FIG. 5b.

In a further embodiment, the corneal batten 10 is coated with a bio-compatible material, acceptable to the ocular environment. Such bio-compatible coating material can include, but is not limited to, PMMA, BIOGLASS, or collagen from an appropriate mammalian source. The corneal batten 10 can be coated with the bio-compatible material using techniques well know in the art, including, dipping in or drawing the corneal batten through liquified bio-compatible material. Additionally, the bio-compatible material can be polymerized on the corneal batten or vapor deposited. The corneal batten 10 can be coated with solutions of the bio-compatible material and then have the solvent evaporated off.

In another embodiment, the bio-compatible coating material promotes cell attachment or contains medications or growth factors which can be controllably released.

In another embodiment, as shown in FIG. 5B, the corneal batten 10 is made by spinning or extruded as composites of smaller diameter fibers. Additionally, the corneal batten 10 can be braided from smaller diameter fibers.

In a method of use, the corneal batten 10 is implanted into the corneal stroma 24. The curvature of cornea 20 is controllably altered by the following: selection of the position of placement of the corneal battens 10; varying the number of corneal battens 10 implanted; varying the dimensions of implanted corneal battens 10; and by flattening or over curving the cornea 20 during placement of the corneal battens 10. The inserted corneal battens 10 will act to alter the stiffness of the surrounding corneal tissue. This increased stiffness will prevent the natural forces acting on the cornea 20 from restoring the original curvature of the cornea 20 and bring about improved visual acuity.

The corneal battens 10 are inserted into the corneal stroma 24 by two actions. First, an incision can be made in the corneal epithelium 26 and Bowman's layer 28 by probing with a sharpened instrument (e.q. scalpel, needle or sharpened cannula) or by pressing the sharpened end of the corneal batten 10 against the corneal epithelium 26 with sufficient force to penetrate the epithelial layer 26 and Bowman's layer 28. Next, the corneal stroma 24 may be bluntly dissected with a probe and the batten 10 may be inserted into the resulting pocket, or the corneal batten 10 itself may be used.

The placement of the corneal batten 10 and the number used is determined by the refractive condition. The initial curvatures and dimensions of the cornea 20 must be ascertained by use of a keratometer or similar instruments. Once these parameters are known, the surgeons would, by consulting tables developed from experimentation, select the number of battens 10 of the desired dimensions and material properties needed to effect the desired alterations. In an example, if the surgeon desired to impart −1.0 diopters of spherical correction to an eye of 7.7 mm radius of curvature and 14.1 mm corneal diameter, the recommended number of battens 10 of 15 microns diameter and 4.0 mm length would be determined from the table and the placement would follow from accepted practice. Measurements could be taken again after a portion of the battens 10 had been placed and corrections could be made, if needed, to the surgical plan.

In an embodiment, a computer program is used to determine the individual dimensions, number required, and the appropriate placement of the corneal battens 10. The algorithms used to generate a table would be incorporated into an interactive computer application. The dimensions and measurements would be entered by hand or taken into the computer directly from the measuring equipment and the program would then make recommendations based on this input. As before, measurements could be taken again after one or more of the battens had been implanted and correction could be made, if needed, to the surgical plan.

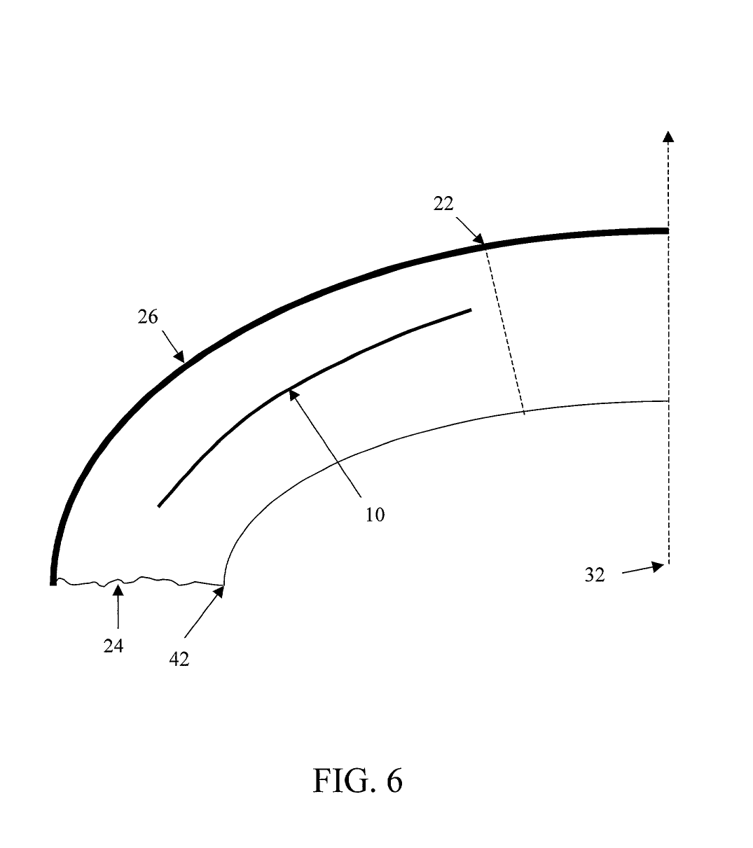

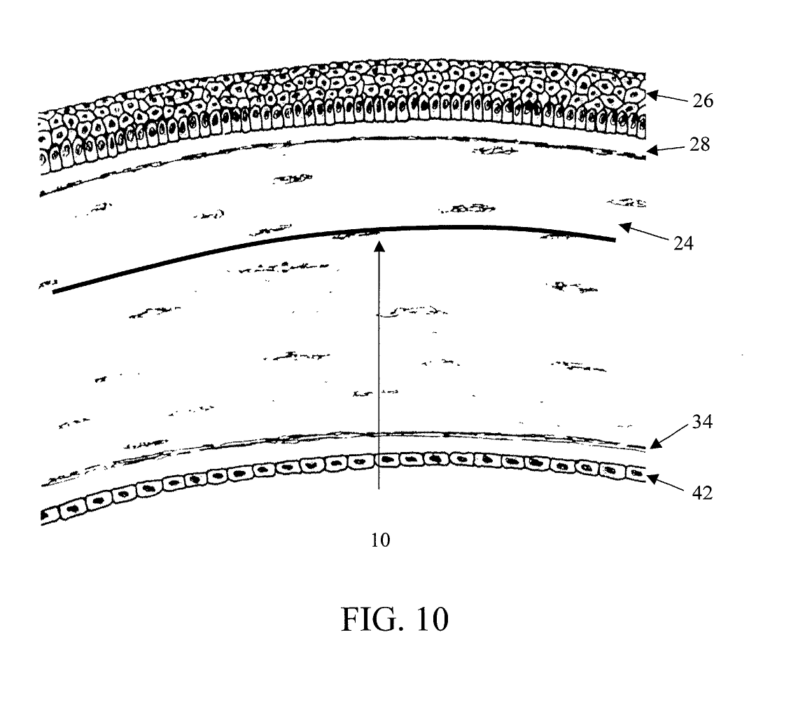

In an embodiment, as shown in FIGS. 6, 7 and 10, the radius of curvature of the optic zone 22 is decreased by inserting at least one corneal batten 10 between the layers of the corneal stroma 24 in the limbal region 30. Where the long axis of the corneal batten 10 is oriented radially to the center of the cornea or pupillary axis 32. In an embodiment the limbal region 30 of the cornea 20 is flattened with a probe element to facilitate the insertion of the corneal batten 10 between discrete layers of the corneal stroma 22. The probe would consist of a flat surface mounted on a suitable holder. The flat surface of the probe would be pressed against that portion of the corneal limbal region 30 wherein the corneal batten 10 is to be inserted. With the cornea 20 flattened, the surgeon would then insert the corneal batten 10 parallel to the flat surface of the probe and a fixed distance from it (e.g. 100 to 400 microns, to be below Bowman's layer 28 and above Descemet's membrane 34). With the overall corneal diameter unaltered, the optic zone 22 must take on a smaller radius of curvature to compensate for the increased radius of curvature of the limbal region 30 that was stiffened by the corneal batten 10.

In an alternative embodiment, the flat surface of the probe could be mounted onto the insertion tool for the corneal batten to facilitate use and insure parallelism.

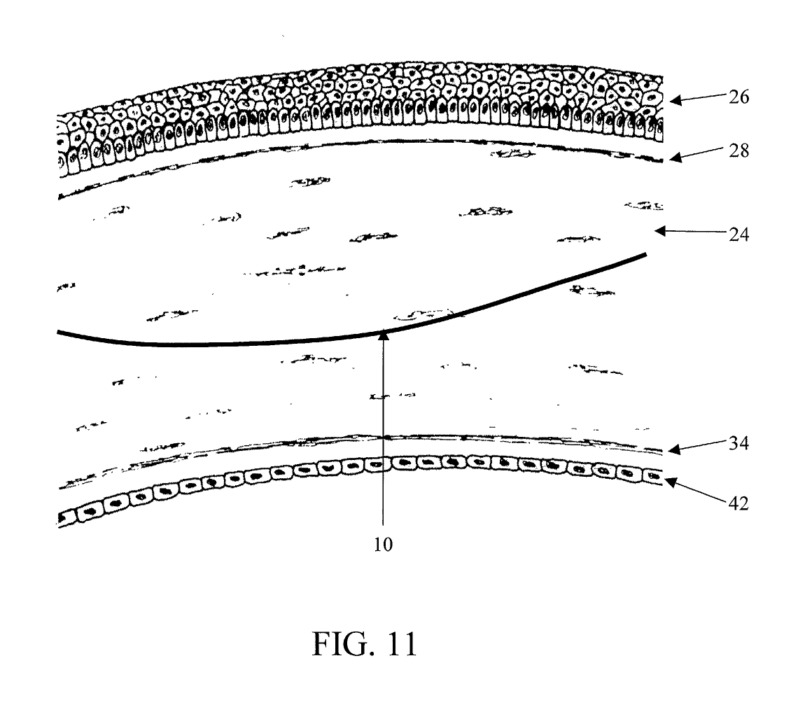

In an embodiment, as shown in FIGS. 9 and 11, the radius of curvature of the optic zone 22 is decreased by inserting at least one corneal batten 10 so as to penetrate several layers of the corneal stroma 24 in the limbal region 30. Where the long axis of the corneal batten 10 is oriented tangentially to the center of the cornea and perpendicular to the pupillary axis 32. In an embodiment the entire limbal region 30 is rendered more conical in shape with a probe element during the insertion of the corneal batten 10 to facilitate the corneal batten 10 penetrating multiple layers of the corneal stroma 24. The probe element would consist of a ring of inner diameter slightly less than the diameter of the exterior surface of the cornea where it intersects the plane on which it is desired to pace the corneal batten. The ring would be mounted on a suitable holder. The ring would be pressed against the cornea 20, causing the optic zone 22 to protrude through the center of the ring. This protrusion causes the cornea 20 to take on a smaller radius of curvature. The surgeon then inserts one or more corneal battens 10 in the same plane as the ring. The batten(s) 10 are inserted as a chord to the external curvature of the cornea 20 and, thus, would penetrate multiple layers of the stroma 24. With the corneal stroma 24 stiffened by the batten(s) 10, the increased stiffness will prevent the natural forces acting on the cornea 20 from restoring the original curvature.

In an alternative embodiment, the ring is mounted on the insertion tools for the corneal battens to facilitate ease of use and insure co-planarity.

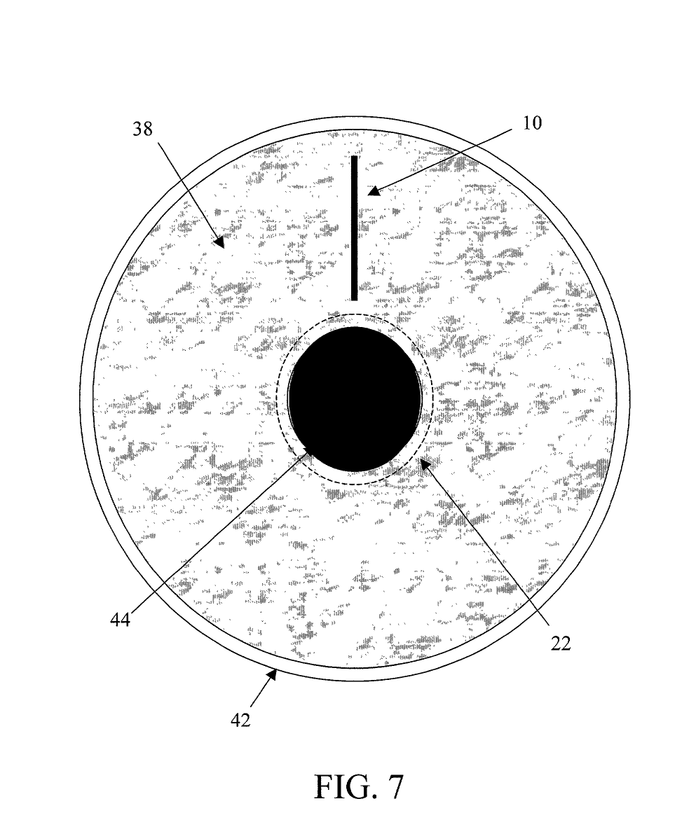

In an embodiment, as shown in FIGS. 7 and 11, the radius of curvature of the optic zone 22 is increased by inserting at least one corneal batten 10 so as to penetrate several layers of the corneal stroma 24 in the limbal region 30. The long axis of the corneal batten is oriented radially to the center of the cornea or the pupillary axis 32. In this embodiment, the optic zone 22 is flattened with a probe element. The flattening of the optical zone 22 creates a smaller radius of curvature in the limbal region 30, facilitating the insertion of the corneal batten 10 through multiple layers of the corneal stroma 24. The probe consists of a flat surface mounted on a suitable holder. The flat surface of the probe is pressed against the optic zone 22. The probe is sufficiently small or possesses an opening so as not to obscure that portion of the corneal limbal region 30 wherein the corneal batten 10 is to be inserted. With the optic zone 22 flattened, the surgeon inserts the corneal batten 10 at an angle to the flat surface of the probe and so as to be a chord to the exterior curvature of the limbal region 30. With the overall corneal diameter unaltered, the optic zone 22 must take on a large radius of curvature to compensate for the decreased radius of curvature of the limbal region 30 that was stiffened by the corneal batten 10.

In an embodiment, the flat surface of the probe is mounted onto the insertion tool for the corneal batten to facilitate use and insure precision of placement

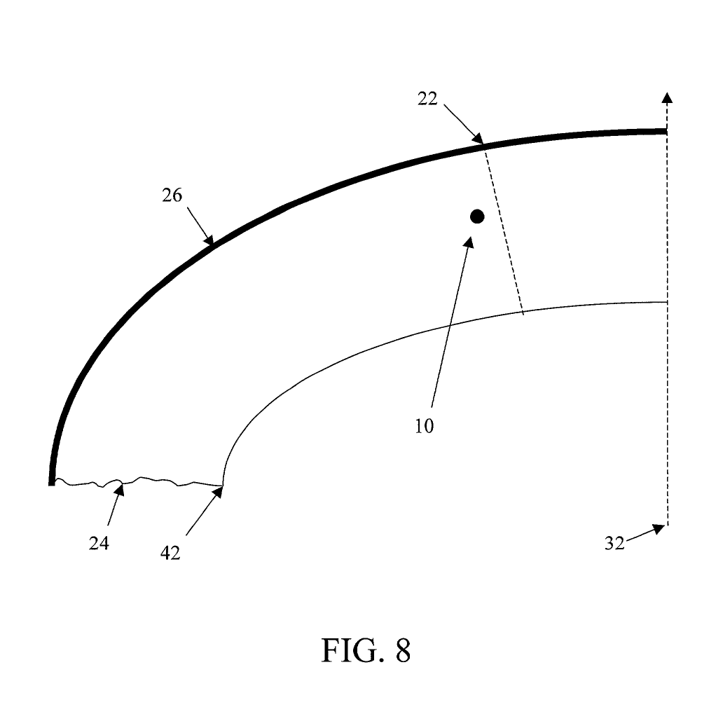

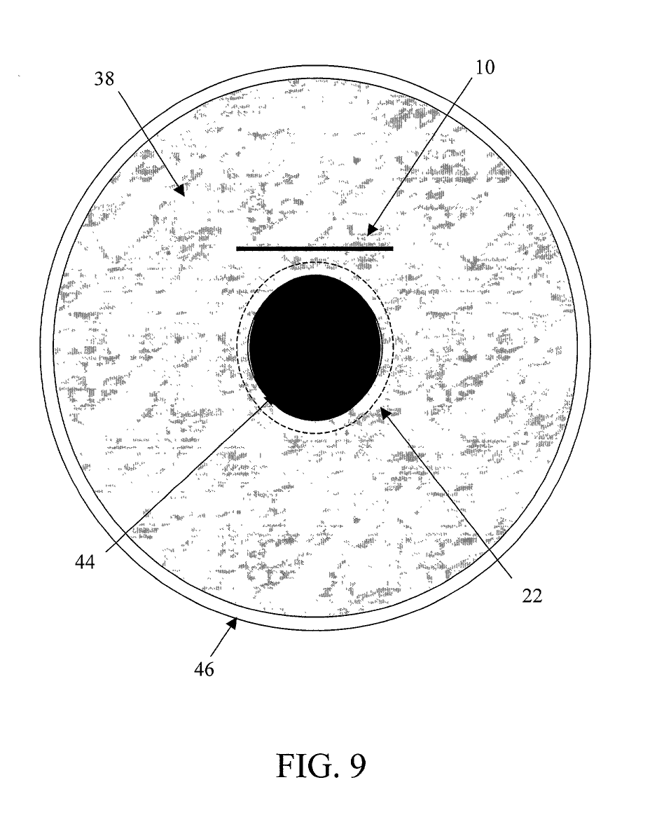

In an alternative embodiment, as shown in FIGS. 8, 9 and 10, the radius of curvature of the optic zone 22 is increased by inserting at least one corneal batten 10 between layers of the corneal stroma 24 in the limbal region 30. The long axis of the corneal batten 10 is oriented tangentially to the center of the cornea and perpendicular to the pupillary axis 32. In this embodiment the limbal region 30 of the cornea 20 is flattened with a probe element to facilitate the insertion of the corneal batten 10 between discrete layers of the corneal stroma 24. The probe consists of a flat surface mounted on a suitable holder. The flat surface of the probe is pressed against that portion of the corneal limbal region 30 wherein the corneal batten 10 is to be inserted. With the cornea 20 flatted, the surgeon inserts the corneal batten 10 parallel to the flat surface of the probe and a fixed distance from the probe (e.g. 100 to 400 microns, to be below Bowman's Layer 28 and above Descemet's membrane 34); tangential to the optic zone 22; and perpendicular to the pupillary axis 32. The corneal stroma 24 is stiffened by the batten 10 and this increased stiffness acts against the natural forces on the cornea 20 that would restore the original curvature. With the overall corneal diameter unaltered, the optic zone 22 must also take on a larger radius of curvature to accommodate the increased radius of curvature to the limbal region 30, tangential to the optic zone 22, that was stiffened by the corneal batten.

In an alternative embodiment, the flat surface of the probe is mounted onto the insertion tool for the corneal batten to facilitate use and insure parallelism.

In a further embodiment a suction cup-form is used to flatten or accentuate the curvature in the region in which the fiber is being inserted. In each of the embodiments discussed previously, the probe that was pressed against the cornea 20 by the surgeon is replaced by a cup possessing the appropriately shaped inner surface. The cup is placed on the cornea 20 and evacuated. The resulting suction pulls the cornea 20 against the inner surface of the cup and the corneal batten(s) 10 are inserted thought appropriately placed ports in the wall to the cup. The cup maybe mounted onto the insertion tool for the cornea 20 to facilitate ease of use and precision of placement.

Following are examples which illustrate procedures for practicing the invention. These examples should not be construed as limiting.

### EXAMPLE 1

### Decrease the Radius of Curvature of the Optic Zone

The radius of curvature of the optic zone 22 can be decreased by flattening the radius of curvature of the limbal region 30 of the cornea 20. A portion of the limbal region 30 would be flattened with a probe and a corneal batten 10 would be inserted through the anterior surface along a line that extends radially from the edge of the optic zone 22 towards the limbus 46. The corneal batten 10 would be inserted at a depth that places it below the Bowman's layer 28 (e.g., 100 microns into the stroma 24) and parallel to the flattened anterior surface. When the probe is removed, the limbal region 30, along the line of the corneal batten 10 will have a higher composite modulus and, thus, will not return to as small a radius of curvature. The resulting distortion in the limbal region 30 must be compensated for by a decrease in the radius of curvature in the optic zone 22 (along the line of the corneal batten 10). As more battens 10 are added around the circle of the optic zone 22, the more uniform is the change in curvature inside the optic zone 22.

In a preferred embodiment the corneal 10 is of ca. 7 microns diameter and ca. 4 mm length. The corneal batten 10 is made from a material possessing a Young's modulus in excess of 100×10⁶ Pascal (1 Pascal-1 Newton/square meter). Such a material includes, but not limited to graphite fiber.

### EXAMPLE 2

### Flattening the Optic Zone Radius of Curvature

The radius of curvature of the optic zone 22 can be flattened by decreasing the radius of curvature of the limbal region 30 of the cornea 20. To do this, the optic zone 22 would be flattened with a probe and a graphite fiber of ca. 7 microns diameter and ca. 4 mm length would be inserted through the anterior surface along a line that extends radially from the edge of the optic zone 22 towards the limbus 46. The fiber would be inserted at a depth that places it well below the Bowman's layer 28 and along a line that would lie on the chord for the external curvature of the limbal region 30. When the probe is removed, the limbal region 38 along the line of the fiber will have a higher composite modulus and, thus, will not return to as large a radius of curvature. This distortion in the limbal region 30 must be compensated for by an increase in the radius of curvature in the optic zone 22 (along the line of the fiber). As more fibers are added around the circle of the optic zone 22, the more uniform is the change in curvature inside the optic zone 22.

It should be understood that the examples and embodiments described herein are for illustrative purposes only and that various modifications or changes in light thereof will be suggested to persons skilled in the art and are included within the spirit and purview of this application and the scope of the appended claims.

I claim:

1. A device for controllably altering the curvature of the cornea comprising a corneal batten, wherein said corneal batten comprises at least one elongated fiber, and where said corneal batten is implanted into the corneal stroma of an eye.

2. The corneal batten according to claim 1, wherein said corneal batten has a length of about 0.1 mm to 16 mm.

3. The corneal batten according to claim 2, wherein said corneal batten has a length of about 2 mm to 6 mm.

4. The corneal batten according to claim 1, wherein said corneal batten has a cross sectional dimension of about 0.1 microns to 300 microns.

5. The corneal batten according to claim 4, wherein said corneal batten has a cross sectional dimension of about 1 micron to 25 microns.

6. The corneal batten according to claim 1, wherein said cross sectional dimension is circular, elliptical, rectangular or triangular.

7. The corneal batten according to claim 1, wherein said cross sectional is hollow.

8. The corneal batten according to claim 1, wherein said corneal batten is made of a material having a Young's modulus greater then the Young's modulus of the corneal stroma.

9. The corneal batten according to claim 1, wherein said corneal batten is made of a bio-compatible material.

10. The corneal batten according to claim 1, wherein said corneal batten is coated with a bio-compatible material.

11. The corneal batten according to claim 1, wherein said corneal batten is formed by a method selected from the group consisting of extruding, spinning, and braiding said fibers.

12. A method for controllably altering the curvature of a cornea comprising the following steps;
a) determining the curvature of the cornea;
b) selecting at least one corneal batten of suitable dimension for correction of the curvature; and
c) inserting said corneal batten into the corneal stroma.

13. The method for controllably altering the curvature of the cornea according to claim 12, further comprising the step of inserting at least one of said corneal battens between the layers of the corneal stroma in the limbal region.

14. The method for controllably altering the curvature of the cornea according to claim 12, wherein said corneal batten is inserted outside of the optic zone.

15. The method for controllably altering the curvature of the cornea according to claim 12, wherein said corneal batten is inserted inside the optic zone.

16. The method for controllably altering the curvature of the cornea according to claim 13, wherein said corneal batten has a longitudinal length which is oriented radially to a center of the cornea, to decrease the radius of curvature of the optic zone.

17. The method for controllably altering the curvature of the cornea according to claim 13, wherein said corneal batten has a longitudinal length which is oriented tangentially to the optic zone and perpendicular to the pupillary axis, to increase the radius of curvature of the optic zone.

18. The method for controllably altering the curvature of the cornea according to claim 12, further comprising the step of inserting at least one of said corneal batten through several layers of the corneal stroma in the limbal region.

19. The method for controllably altering the curvature of the cornea according to claim 18, wherein said corneal batten has a longitudinal length which is oriented tangential to the optic zone and perpendicular to the pupillary axis, to decrease the radius of curvature of the optic zone.

20. The method for controllably altering the curvature of the cornea according to claim 18, wherein said corneal batten has a longitudinal length which is oriented radially to a center of the cornea, to increase the radius of curvature of the optic zone.

21. The method for controllably altering the curvature of the cornea according to claim 12, further comprising the step of altering the shape of the limbal region with a probe element prior to inserting said corneal batten.

22. The method for controllably altering the curvature of the cornea according to claim 21, wherein said probe element flattens the limbal region of the cornea prior to inserting said corneal batten.

23. The method for controllably altering the curvature of the cornea according to claim 21, wherein said probe element increases the conical shape of the limbal region of the cornea prior to inserting said corneal batten.

24. A method for effecting refractive correction by locally altering the stiffness of the cornea, comprising the following steps;
a) measuring the curvature of the cornea;
b) determining at least one correction location on the cornea;
c) selecting at least one corneal batten of suitable dimension; and
d) insertion said corneal batten into the corneal stroma at said correction location.

25. A computer implemented method of controllably altering the curvature of the cornea, comprising the following steps;
a) measuring the curvature of the cornea;
b) calculating at least one correction location on the cornea for curvature alternation;
c) determining the quantity and dimensions of said corneal battens; and
d) insertion said corneal battens into the corneal stroma at said correction locations.
